(19) United States
(12) Patent Application Publication
(10) Pub. No.: US 2015/0104501 A1
(43) Pub. Date: Apr. 16, 2015

(54) LIPID-SUPPORTED POLYMERIC FUNCTIONAL PARTICLES AND METHOD OF PRODUCING THE SAME

(71) Applicant: RESEARCH & BUSINESS FOUNDATION SUNGKYUNKWAN UNIVERSITY, Suwon-si (KR)

(72) Inventors: Soong Ho UM, Seoul (KR); Seung Won SHIN, Seoul (KR)

(73) Assignee: RESEARCH & BUSINESS FOUNDATION SUNGKYUNKWAN UNIVERSITY, Suwon-si (KR)

(21) Appl. No.: 14/513,970

(22) Filed: Oct. 14, 2014

(30) Foreign Application Priority Data
Oct. 15, 2013 (KR) 10-2013-0122934

(51) Int. Cl. A61K 9/127

(52) U.S. Cl. CPC A61K 9/1273; A61K 9/1277
USPC 424/450; 514/1.1; 514/44 R

(57) ABSTRACT

The present disclosure relates to functional composite particles produced by filling water-soluble or lipid-soluble polymers into changeable liposomes and a method of producing the same. The present disclosure also relates to an evaluation of specialized biochemical characteristics of composites after the composites are produced using the water-soluble or lipid-soluble polymers by selection from groups capable of combining with lipid layers. A protocol according to an embodiment of the present disclosure overcomes the limitations of a conventional water/oil-based single emulsion protocol to prepare single polymer particles or single lipid layered particles and combine liposomes with a variety of polymer groups.

### CROSS-REFERENCE TO RELATED APPLICATION

This application claims the benefit under 35 USC 119(a) of Korean Patent Application No. 10-2013-0122934, filed on Oct. 15, 2013, in the Korean Intellectual Property Office, the entire disclosure of which is incorporated herein by reference for all purposes.

### BACKGROUND

1. Field

The following description relates to functional particles of liposomes in which water-soluble polymers or lipid-soluble polymers are combined, and a method of producing the same.

2. Discussion of Related Art

Liposomes are spherical vesicles in which a phospholipid bilayer surrounds an aqueous phase filling an inner space of the vesicle. Constituents of lipid layers are amphipathic phospholipids each comprising two hydrophobic fatty acid groups and a hydrophilic phosphate group. When exposed to an aqueous phase, the phospholipids arrange themselves into a bilayer that may form a closed structure such as an artificial cell. In a bilayer structure, hydrophobic lipid tails face the inside of the layer while the hydrophilic heads face the outside thereof. A drug injected into the liposomes exhibit decreased toxicity and increased pharmaceutical efficacy. Therefore, the liposomes are receiving attention as a particle structure prepared through assembly with polymers, drugs, and antigens.

However, there are many issues associated with the single emulsion protocol that are used in the process of producing single polymer particles or single lipid particles for use as a carrier according to a conventional art. The single emulsion protocol involves using a bilayer of water/oil. For instance, this conventional method only allows the use of a lipid-soluble polymer, which largely limits a range of available polymers when used for actual medical treatments. Further, clinical adaptations have exposed the limitations of unilamellar lipid particles which are easily decomposed.

Particularly, substances generated when polymers are exposed or decomposed in cells and tissues commonly damage surrounding normal cells or cause side effects such as inflammatory responses. On the other hand, water-soluble polymers are usually present naturally, and thus may have the benefit of minimizing an adverse effect. Since water-soluble polymers such as polysaccharides, polydeoxyribonucleic acids, collagen, and cellulose are all present naturally and may be included into cell metabolites, side effects may be minimized upon decomposition of the water-soluble polymers.

However, when water-soluble proteins are entrapped in particles, severe side effects occur upon the production and application of such particles, such as aggregation of most proteins on oils. Accordingly, particles may be formed only at a predetermined concentration of the entrapped proteins. Due to a limitation of available polymers and difficulty in treating proteins, there are many difficulties in application of liposomes to living bodies such as producing immune vaccines based on protein antigens or antibodies.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

According to an aspect of the present disclosure, there is provided a particle in which a polymer and a drug are combined in a liposome formed of a lipid.

In an embodiment of the present disclosure, the polymer may be a water-soluble polymer or a lipid-soluble polymer.

In an embodiment of the present disclosure, when the polymer is the water-soluble polymer, a concentration of a lipid is in a range of 1 to 10 mM.

In an embodiment of the present disclosure, when the polymer is the lipid-soluble polymer, a concentration of a lipid is in a range of 3 to 5 M.

In an embodiment of the present disclosure, the water-soluble polymer is one or more selected from the group consisting of polydeoxyribonucleic acids, agaroses, alginates, carrageenans, hyaluronic acids, dextrans, chitosans, and cyclodextrins.

In an embodiment of the present disclosure, the lipid-soluble polymer is one or more selected from the group consisting of polylactides, polyglycolides, poly-gamma-glutamic acid (BLS-PGA), polycaprolactones, polyethylene glycol, poly(hydroxy butyrate), poly(ε-caprolactone), poly(β-malic acid), poly(lactic acid-co-glycolic acid) and mixtures thereof.

In an embodiment of the present disclosure, the lipid-soluble polymer is one or more selected from the group consisting of polylactides, polyglycolides and mixtures thereof.

In an embodiment of the present disclosure, a mole ratio of a polylactide and a polyglycolide is 25˜75:75˜25 in the mixture.

In an embodiment of the present disclosure, the lipid is one or more selected from the group consisting of 1,2-dioleoyl-sn-glycero-3-phosphocholine (DOPC), 1,2-dioleoyl-sn-glycero-3-phospho-(1′-rac-glycerol) sodium salt (DOPG), 1,2-dioleoyl-sn-glycero-3-phosphoethanolamin-N-[4-(p-maleimidophenyl)butyramide] (MPB-PE), 1,2-dihexadecanoyl-sn-glycero-3-phosphoethanolamine, triethylammonium salt (Texas Red DHPE), cholesterol, lecithin, and mixtures thereof.

In an embodiment of the present disclosure, the drug is ovalbumin or CpG oligodeoxynucleotide.

In an embodiment of the present disclosure, a diameter of the particle is in a range of 200 to 1,500 nm.

According to another aspect of the present disclosure, there is provided a method of producing particles in which the water-soluble polymers are combined, including:

a) mixing water-soluble polymers and lipids;

b) preparing an emulsion by stirring the mixed solution or treating the mixed solution with ultrasonic waves; and

c) removing an organic solvent positioned in an upper layer of the emulsion by centrifugation of the emulsion.

In an embodiment of the present disclosure, the water-soluble polymer is one or more selected from the group consisting of polydeoxyribonucleic acids, agaroses, alginates, carrageenans, hyaluronic acids, dextrans, chitosans, and cyclodextrins.

In an embodiment of the present disclosure, a drug is further mixed in step a).

In an embodiment of the present disclosure, the drug is ovalbumin or CpG oligodeoxynucleotide.

In an embodiment of the present disclosure, the lipid is one or more selected from the group consisting of 1,2-dioleoyl-sn-glycero-3-phosphocholine (DOPC), 1,2-dioleoyl-sn-glycero-3-phospho-(1′-rac-glycerol) sodium salt (DOPG), 1,2-dioleoyl-sn-glycero-3-phosphoethanolamin-N-[4-(p-maleimidophenyl)butyramide] (MPB-PE), 1,2-dihexadecanoyl-sn-glycero-3-phosphoethanolamine, triethylammonium salt (Texas Red DHPE), cholesterol, lecithin, and mixtures thereof.

According to still another aspect of the present disclosure, there is provided a method of producing particles in which the lipid-soluble polymer is combined, including:

a) mixing lipid-soluble polymers and lipids;

b) preparing a single emulsion by treating the mixed solution with ultrasonic waves;

c) preparing a multiple emulsion by adding an aqueous solution including a drug to the single emulsion and treating the single emulsion with ultrasonic waves;

d) removing an organic solvent by stirring the multiple emulsion; and

e) centrifuging the emulsion from which the organic solvent is removed in step d).

In an embodiment of the present disclosure, the lipid-soluble polymer is one or more selected from the group consisting of polylactides, polyglycolides, poly-gamma-glutamic acid (BLS-PGA), polycaprolactones, polyethylene glycol, poly(hydroxy butyrate), poly(ε-caprolactone), poly(β-malic acid), poly(lactic acid-co-glycolic acid) and mixtures thereof.

In an embodiment of the present disclosure, the lipid-soluble polymer is one or more selected from the group consisting of polylactides, polyglycolides and mixtures thereof.

In an embodiment of the present disclosure, a mole ratio of a polylactide and a polyglycolide is 25˜75:75˜25 in the mixture.

In an embodiment of the present disclosure, the lipid is one or more selected from the group consisting 1,2-dioleoyl-sn-glycero-3-phosphocholine (DOPC), 1,2-dioleoyl-sn-glycero-3-phospho-(1′-rac-glycerol) sodium salt (DOPG), 1,2-dioleoyl-sn-glycero-3-phosphoethanolamin-N-[4-(p-maleimidophenyl)butyramide] (MPB-PE), 1,2-dihexadecanoyl-sn-glycero-3-phosphoethanolamine, triethylammonium salt (Texas Red DHPE), cholesterol, lecithin, and mixtures thereof.

In an embodiment of the present disclosure, the drug is ovalbumin or CpG oligodeoxynucleotide.

Other features and aspects will be apparent from the following detailed description, the drawings, and the claims.

### BRIEF DESCRIPTION OF THE DRAWINGS

FIG. 1 is a view illustrating a structure of complex particle filled with a lipid-soluble polymer according to an example of the present disclosure.

FIG. 2 shows images obtained using a transmission electron microscope and a confocal microscope through which a particle formation according to one example can be determined.

FIG. 3 shows tables showing a size and a surface charge of the produced particles according to one example. Table (a) relates to physical properties of particles in which lipid-soluble polymer groups are included, and table (b) shows physical properties of particles in which water-soluble polymers (nucleic acids) at various concentrations are included.

FIG. 4 shows tables showing an amount of drugs entrapped in particles according to one example. Table (a) demonstrates a degree of injecting a nucleic acid and ovalbumin in particles with respect to lipid-soluble polymer groups, and table (b) demonstrates a degree of injecting a water-soluble polymer in particles;

FIG. 5 shows tables for comparison of determination whether particles are formed with a uniform size by applying ovalbumin of various concentrations to particles prepared by an existing method and particles prepared by a method according to an example of the present disclosure. Table (a) shows a size of particles prepared by an existing method, and table (b) shows a size of particles prepared by a method according to an embodiment of the present disclosure;

FIG. 6 is a graph that demonstrates a decomposition rate difference between PLGA 50:50 (lactide:glycolide), PLGA 75:25 (lactide:glycolide), and PLA (lactide only) used as representative lipid-soluble polymer groups in one example.

FIG. 7 is an electrophoresis image that demonstrates that a nucleic acid used as a representative water-soluble polymer group forms a structure in a particle by ligase.

FIG. 8 is confocal microscope images showing a nucleic acid gel that is a water-soluble polymer and positioned within a particle.

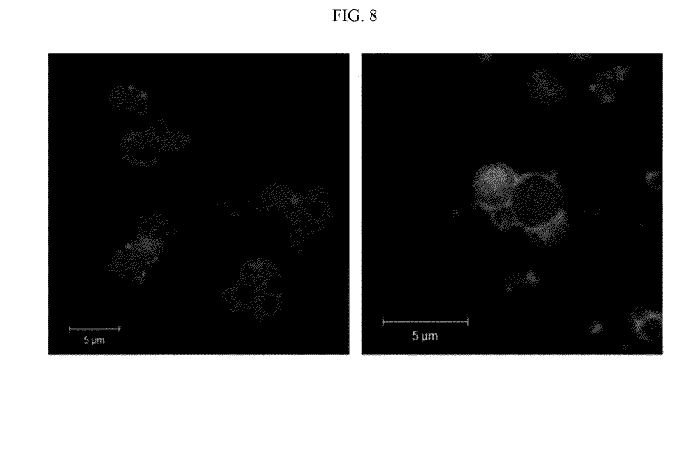

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

While the single emulsion protocol only allows the use of a lipid-soluble polymer, it is beneficial to use lipid-soluble and water-soluble polymers without limitation according to a type and feature of a target disease, as well as to inject a desired drug into the liposomes. In order to address such issues, in the present disclosure, an example of an innovative method of preparing composites having lipid layers in which polymers are entrapped and using them as a functional drug carrier is provided.

One example of the present disclosure is directed to a method of producing particles by entrapping water-soluble and lipid-soluble polymers into liposomes and enabling drugs to be injected into the particles without limitation. However, the technical objectives of the present disclosure are not limited to the same and other objectives may become apparent to those of ordinary skill in the art based on the following descriptions.

The present disclosure was completed as a result of a study on multifunctional particles prepared by combining polymers having various features into changeable liposomes.

Accordingly, the present disclosure is directed to providing liposome particles in which polymers are combined and drugs are included.

That is, the present disclosure may provide multifunctional particles by filling water-soluble polymers or lipid-soluble polymers into changeable liposomes and injecting desired drugs therein.

In another embodiment of the present disclosure, the polymer combined in the liposome particles of the present disclosure may be a water-soluble polymer or a lipid-soluble polymer.

As a result of an experiment liposomes prepared using lipids of various compositions and amounts in order to form liposomes, it was determined that it is preferable to use lipids in a range of a total of 3 to 5 M in the case of the lipid-soluble polymer, and lipids in a range of a total of 1 to 10 mM in the case of the water-soluble polymer. Further, it was determined that it is more preferable to use lipids of a total of 4.2 M in the case of the lipid-soluble polymer, and lipids of a total of 5.54 mM in the case of the water-soluble polymer in order to form particles.

In another embodiment of the present disclosure, 1,2-dioleoyl-sn-glycero-3-phosphocholine (DOPC), 1,2-dioleoyl-sn-glycero-3-phospho-(1′-rac-glycerol) sodium salt (DOPG), 1,2-dioleoyl-sn-glycero-3-phosphoethanolamin-N-[4-(p-maleimidophenyl)butyramide] (MPB-PE), 1,2-dihexadecanoyl-sn-glycero-3-phosphoethanolamine, triethylammonium salt (Texas Red DHPE), cholesterol, lecithin, or a mixture thereof may be used as the lipid, but the lipid is not limited thereto, and a composition and an amount thereof may be subdivided as necessary.

Among the above lipids, Texas Red DHPE including a fluorescent material or MPB-PE specifically combining with a thiol group (—SH) is desirable for providing multifunctionality to particles. Texas Red DHPE is a lipid that is commercially produced and sold, and is known to emit light in a wavelength of 615 nm in response to light in a wavelength of 595 nm. It was determined that a fluorescence image of particles may be obtained by adding 0.02 to 0.03 mg of Texas Red DHPE to conventional lipid compositions.

A lipid composition is not largely involved in particle formation, but causes changes in simple surface properties. As a result of an experiment of varying a surface lipid composition in order to contribute to multifunctionality of particles, since a total charge is formed to be −1 per molecule in the case of DOPG among the above lipids, it was determined that a surface charge gradually turns into a negative charge when a fraction of DOPG is increased. Since a surface charge of a cell is a negative charge, negative poling of a surface charge of a particle is highly important in an experiment of actual cells and tissues. Therefore, it may be determined that surface properties of particles may be variously controlled by changing a lipid composition in formation of the particles according to the embodiment of the present disclosure.

In still another embodiment of the present disclosure, the water-soluble polymers may be polydeoxyribonucleic acids, agaroses, alginates, carrageenans, hyaluronic acids, dextrans, chitosans, or cyclodextrins, and most preferably, polydeoxyribonucleic acids.

The polydeoxyribonucleic acids are basically the same as nucleic acids that form genes within cells. A polydeoxyribonucleic acid used in the embodiment of the present disclosure is produced such that a composition unit includes four ends and forms an X shape, and a combination of each polydeoxyribonucleic acid is formed through a temporary crosslinking of the complementary four ends (overhangs) and through ligase in which the crosslinking is substituted with a covalent bond. Thus, water-soluble polymeric particles capable of maintaining a shape of the structures even after liposomes are decomposed may be formed.

The lipid-soluble polymer may be a polylactide, a polyglycolide, poly-gamma glutamic acid (BLS-PGA), a polycaprolactone, polyethylene glycol, poly(hydroxy butyrate), poly(ε-caprolactone), poly(β-malic acid), poly(lactic acid-co-glycolic acid) or a mixture thereof. As the lipid-soluble polymer, d,l-lactide/glycolide, which is a mixture of a lactide and a glycolide, is preferably used such as the embodiment of the present disclosure, and a decomposition rate of the particles in a body may vary depending on a composition ratio of the lactide and the glycolide.

In the embodiment of the present disclosure, PLGA 50:50, PLGA 75:25, and PLA, in which composition ratios of lactides and glycolides are respectively 50:50, 75:25, and 100:0, were used. These polymers were included into particles according to the same production method, and it was determined from an experimental result that a drug release time according to a decomposition rate was changed. That is, it was determined that the features of the particles in which lipid-soluble polymers are combined in liposomes according to the embodiment of the present disclosure may be applicable to the effect of multiple inoculations to be achieved through a single inoculation, as an alternative method to multiple inoculations of a vaccine used to establish a strong immune system.

In still another embodiment of the present disclosure, a variety of drugs may be included in particles. The drugs may include both lipid-soluble drugs and water-soluble drugs, and in the embodiment of the present disclosure, an antigen or a nucleic acid which is immunogenic and used as an immunity-inducing model was used as the drugs for application of particles as a multifunctional vaccine platform. Ovalbumin obtained from an egg was used as the antigen, and CpG oligodeoxynucleotide (CpG ODN) known to stimulate TLR 9 was used as the nucleic acid. Preparation of normal particles was determined to be possible from experiments of injecting ovalbumin into particles at various concentrations in the case of a particle filled with a lipid-soluble polymer

In order to form particles in various sizes in accordance with the embodiment of the present disclosure, particles having a diameter in a range of about 200 to 1,500 nm were produced by adding various amounts of energy through stirring or ultrasonic waves in a process forming a water/oil emulsion of solution containing lipid and drug. It was determined that particles having a diameter in a range of 200 to 800 nm are easily injected into cells, and particles having a diameter in a range of 1,000 to 1,500 nm or more have features optimized to morphological imaging or other imaging of particles.

In the case of a polymer filled in liposome, both a lipid-soluble polymer soluble in chloroform or dichloromethane and a water-soluble polymer soluble in an aqueous layer may be used. As an example of available polymers, polysaccharides such as agaroses, alginates, carrageenans, hyaluronic acids, dextrans, chitosans, or cyclodextrins, polyesters such as poly(lactic acid), poly(glycolic acid), poly(hydroxy butyrate), poly(ε-caprolactone), poly(β-malic acid), or poly(lactic acid-co-glycolic acid), and so forth may be used, but it is not limited thereto.

That is, the embodiment of the present disclosure may provide a method of producing liposome particles, in which water-soluble polymers are combined, including:

a) mixing water-soluble polymers and lipids;

b) preparing an emulsion by stirring or treating the mixed solution with ultrasonic waves; and

c) removing an organic solvent positioned in an upper layer of the emulsion by centrifugation of the emulsion.

A drug may be further mixed in step a).

Further, the embodiment of the present disclosure may provide a method of producing liposome particles, in which lipid-soluble polymers are combined, including:

a) mixing lipid-soluble polymers and lipids;

b) preparing a single emulsion by stiffing or treating the mixed solution with ultrasonic waves;

c) preparing a multiple emulsion by adding an aqueous solution including a drug to the single emulsion and treating them with ultrasonic waves;

d) removing an organic solvent by stirring the multiple emulsion; and

e) centrifuging the emulsion from which the organic solvent is removed in step d.

In the embodiment of the present disclosure, an organic solvent such as commonly used chloroform or dichloromethane may be used to dissolve the lipid-soluble polymer. When the organic solvent is used, most lipid-soluble polymers may be dissolved, and thus, most commonly used lipid-soluble polymers may be applicable in the production method according to the embodiment of the present disclosure. Accordingly, although a representative lipid-soluble polymer used in the embodiment of the present disclosure is a poly(d,l-lactide/glycolide), any polymer soluble in chloroform or dichloromethane is applicable.

From the above results, multifunctional particles may be prepared through a production method of assemblies in which polymers are combined in lipid layers in accordance with the embodiment of the present disclosure, and it may be anticipated to apply for the purpose of various medical treatments due to the possibility of an excellent application.

Hereinafter, the present disclosure will be described in detail in conjunction with the following embodiments. However, the following embodiments merely exemplify the present disclosure, and the present disclosure is not limited thereto.

### EXAMPLE

### Example 1

### Establishment of New Multiple Emulsion Protocol for Producing Assemblies in which Polymers are Combined in Lipid Layers and Production Method Thereof

1.1 Preparation of Complex Particles Filled with Lipid-Soluble Polymers

In order to prepare assemblies in which polymers are combined in lipid layers, 1,2-dioleoyl-sn-glycero-3-phosphocholine (DOPC), 1,2-dioleoyl-sn-glycero-3-phospho-(1′-rac-glycerol) (sodium salt) (DOPG), cholesterol, 1,2-dihexadecanoyl-sn-glycero-3-phosphoethanolamine, and triethylammonium salt (Texas Red DHPE) were used. DOPC, DOPG, and cholesterol were obtained from Avanti Polar Lipids, Inc., and Texas Red DHPE was obtained from Life Technologies.

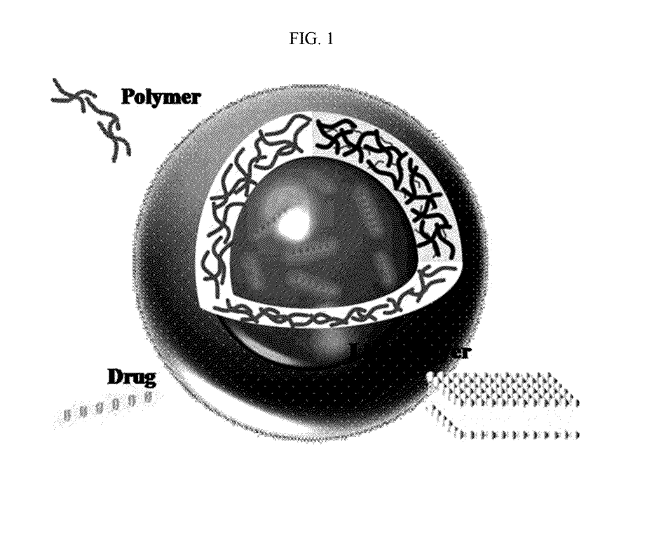

In order to prepare particles having a lipid-soluble polymeric (refer to FIG. 1) using a multiple emulsion solvent evaporation method, 0.03 mg of a polymer and 1.6 mg of a lipid were dissolved in 1 ml of dichloromethane (DCM), and the aqueous solution was dispersed by ultrasonic waves to prepare a first single emulsion. Then, an excess amount of an aqueous solution (6 ml) including the single emulsion and various antigens were mixed by ultrasonic waves at a predetermined interval, and thereby a multiple emulsion was prepared. After the multiple emulsion was formed, the prepared emulsion solution was slowly stirred at room temperature, and the DCM solvent was evaporated and removed completely. Thereafter, polymeric particles dissolved in the solution from which the solvent was evaporated by centrifugation were separated, collected, and washed in water to prepare particles filled with a lipid-soluble polymeric. Physicochemical properties such as a size, a surface charge, and the like of the particles were measured by various devices.

1.2 Preparation of Complex Particles Filled with Water-Soluble Polymers

In order to prepare particles filled with a water-soluble polymeric (e.g., a nucleic acid polymer) layer, a modification and a use thereof are based on a giant unilamellar vesicle formation method. An aqueous solution including X-shaped DNA as a water-soluble polymer used as a block unit and ligation components (T4 ligase and ligase buffer) was mixed with an organic solvent of liquid paraffin or ethyl acetate including a lipid, and an emulsion was formed by stirring or treating the mixed solution with ultrasonic waves. The prepared organic solution including the emulsion was stacked on an aqueous solution, and centrifuged to be separated. Before the centrifugation process, sucrose and glucose were added to an aqueous solution inside a lipid layer to minimize an osmotic pressure. In addition, glucose was added to an aqueous solution outside the particles at the same mole concentration as the sucrose and glucose in the aqueous solution inside the particles in order to minimize an osmotic pressure due to the sucrose and glucose in the aqueous solution inside the particles, and then an upper layer of an organic solvent was removed to prepare particles filled with a water-soluble polymeric. Physicochemical properties such as a size, a surface charge, and the like of the particles were measured by various devices.

1.3 Determination of Particle Formation

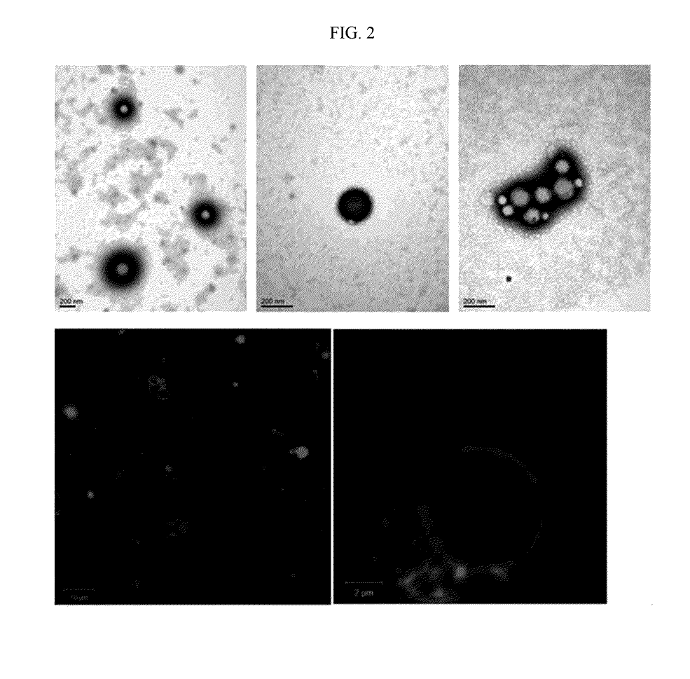

A stabilization of an emulsion and a formation of particles by liposomes were determined with a transmission electron microscope (LIBRA 120) and fluorescence imaging using a confocal microscope (LSM 510) after preparation of the particles. The results are shown in FIG. 2. Upper images in FIG. 2 show images determined using a transmission electron microscope, and lower images in FIG. 2 show images determined using fluorescence imaging.

As shown in FIG. 2, transmission electron microscope images show that particles having a size of about 200 nm were formed smoothly (upper images in FIG. 2), and confocal microscope images show that particles larger than the above particles having a size of 1,000 nm or more were surrounded by lipid surface layers due to the limitation of a decomposition ability (lower images in FIG. 2).

From a combination of the above results, it may be determined that the assemblies having lipid layers in which polymers were combined were formed well using the production method according to the embodiment of the present disclosure.

1.4 Determination of Size and Surface Charge of Prepared Particles

In order to determine an entire size and surface charge of the prepared particles, dynamic light scattering (DLS) was used. A size distribution and surface charge of the particles were measured with lasers in a wavelength of 653 nm using a DLS device (ELS-Z series; manufactured by Otsuka Electronics Co., Ltd.). A size of the particles was measured more precisely using software of the DLS device known as a cumulant method. Here, the number of cumulants was limited to 100 or more for use.

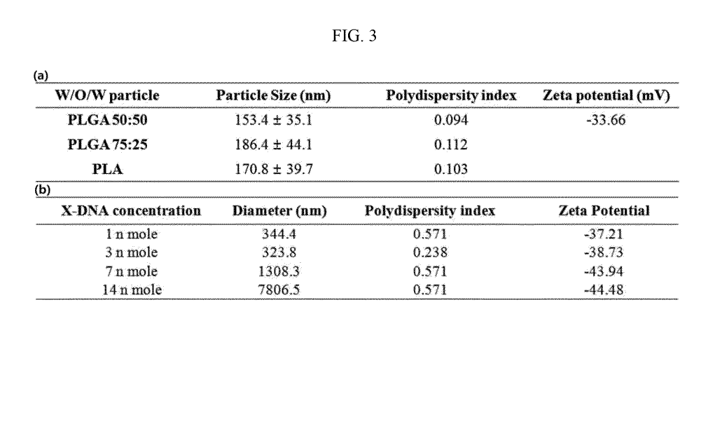

In the case of the particles having a lipid-soluble polymer, an average particle diameter was determined to be 200 nm or less. The results are as shown in FIG. 3(a).

Further, the particles having a water-soluble polymer were also measured with a DLS device, and an average particle diameter was determined to be in a range of 300 to 7800 nm depending on an amount of water-soluble polymer (nucleic acids) filled in the particles. The results are shown in FIG. 3(b).

1.5 Determination of Drug-Entrapment Ability of Prepared Particles

The drug-entrapment of the particles was determined through experiments. An amount of a drug injected into the particles was calculated by measuring a concentration of a drug remaining in a solution after the particles were formed. The drug used herein was largely divided into a nucleic acid (DNA) and ovalbumin (OVA).

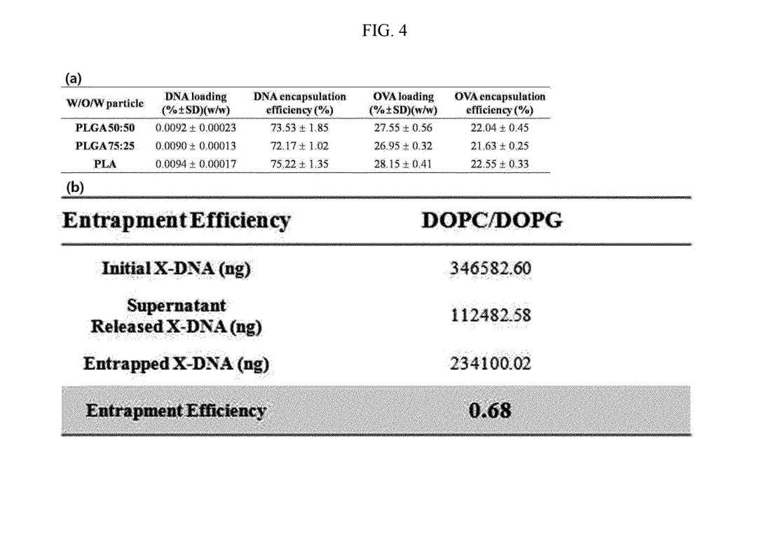

A concentration of the residual nucleic acid was measured using Quant-iT™ PicoGreen® dsDNA Reagent and Kits, and a concentration of ovalbumin was quantified by measuring an Alexa 594 fluorescent material covalently bonded to the ovalbumin. As shown in FIG. 4(a), it was determined that about 73% of the nucleic acid and about 22% of the ovalbumin were injected into the particles having a lipid-soluble polymer.

Further, it was determined that about 68% of the processed nucleic acid in an X shape was injected into the particles having a water-soluble polymer. The results are shown in FIG. 4(b).

1.6 Comparison of Size of Prepared Particles

As determined in the above embodiments, the production method of the embodiment of the present disclosure has an advantage in that it is easier to form particles for a water-soluble protein of a various concentration than the conventional production method for a single polymer particle or a single lipid particle. For a determination, as preparing particles at various protein concentrations using the existing production method and the production method according to the embodiment of the present disclosure for particles having a lipid-soluble polymer, a size of the particle was analyzed and compared.

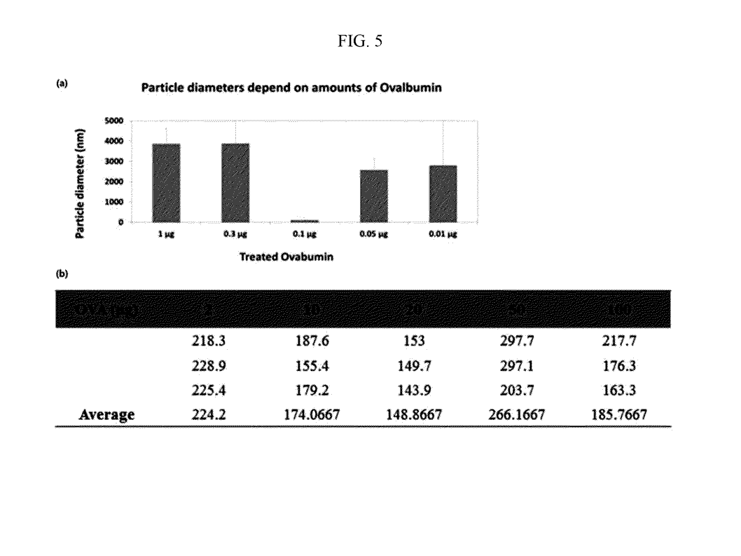

As shown in FIG. 5(a), when the existing production method was used, it was determined that particles having a size of about 200 nm were prepared only at a protein concentration of 0.125 μg/ml.

However, as shown in FIG. 5(b), when the production method according to the embodiment of the present disclosure was used, it was determined that particles having a uniform size of about 200 nm were prepared at all protein concentrations.

### Example 2

### Observation of Change of Particle Properties According to Components and Compositions of (Lipid-Soluble and Water-Soluble) Polymer

2.1 Determination of Change of Particle Properties According to Polymer Components

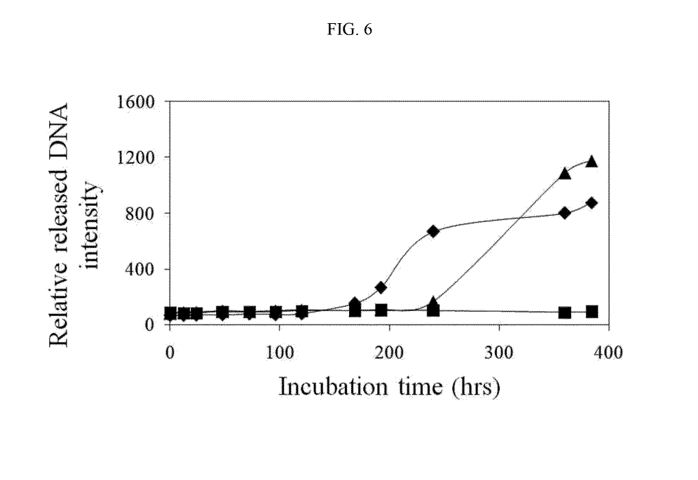

In order to determine diversification of a particle decomposition rate according to a property change of polymers, PLGA 50:50 (lactide:glycolide), PLGA 75:25 (lactide:glycolide), and PLA (lactide only) polymers were used. The same amount, 0.03 mg, of each polymer was used, and nucleic acids were entrapped inside the polymers. The production methods thereof were also the same. After solutions were taken at each measurement time and floating particles were removed therefrom by centrifugation, a nucleic acid concentration of a supernatant liquid was measured using Quant-iT™ PicoGreen® dsDNA Reagent and Kits. The results are shown in FIG. 6.

As shown in FIG. 6, the nucleic acid concentration of the supernatant liquid started to increase in sequence of PLGA 50:50 (lactide:glycolide), PLGA 75:25 (lactide:glycolide), and PLA (lactide only). This means that a particle decomposition proceeded in the sequence of PLGA 50:50 (lactide:glycolide), PLGA 75:25 (lactide:glycolide), and PLA (lactide only). (♦: PLGA 50:50, ▴: PLGA 75:25, ▪: PLA).

From the result, it may be determined that a decomposition rate of particles prepared using the production method according to the embodiment of the present disclosure can be precisely controlled. Accordingly, aspects of the drug release in a body may be regulated using the above characteristics of the particles according to the embodiment of the present disclosure. This may allow a single inoculation to exhibit the same effect as multiple inoculations, as an alternative method to multiple inoculations of a vaccine used to establish a strong immune system.

2.2 Determination of Change of Particle Properties According to Water-Soluble Polymer Components

In the case of a nucleic acid polymer used as a representative water-soluble polymer, it was determined that a nucleic acid which is a basic unit may form a structure by ligase. Therefore, a variety of emulsion formation methods were experimented in the embodiment of the present embodiment, and liquid paraffin was used as an organic solvent in the experiments.

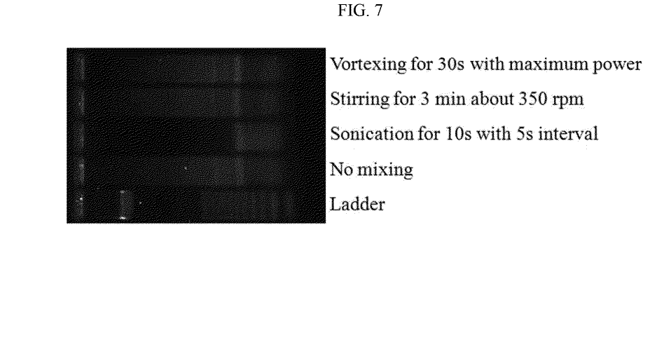

Particles were prepared through an emulsion formation method of vortexing for 30 seconds with maximum power, and then processed using Triton X-100 and surface liposomes were removed. Then, nucleic acids having a molecular weight equal to or more than a basic unit were detected through electrophoresis. The results are shown in FIG. 7.

As shown in FIG. 7, it was determined that a giant nuclear structure was formed in the particle. This shows that an activity of ligase was maintained, and proves that an actual nucleic acid structure was formed.

2.3 Determination of Change of Particle Properties According to Water-Soluble Polymer Components

A nucleic acid gel was used as a water-soluble polymer, and an image of particles filled with a nucleic acid gel was taken using a confocal microscope (LSM 510). Lipid layers were selectively dyed with Texas Red DHPE, and nucleic acids were selectively dyed with SYBR-green I, which is known to dye nucleic acids without affecting an activity of ligase. Thus, it may be determined that a nucleic acid gel, which is a water-soluble polymer, was positioned inside the particle.

In the method of producing particles according to the embodiment of the present disclosure, a new double emulsion method improved from a single emulsion method and a method of forming unilamellar lipid particles filled with polymers allow a functional composite structure having a lipid surface layer and a polymer-antigen combination to be designed and produced. Particles filled with water-soluble polymer may also be prepared easily because the limitation of a single emulsion method in which only a lipid-soluble polymer can be filled is overcome. Accordingly, a range of available polymers may be widened so that lipid-soluble polymers (e.g., PLGA) and water-soluble polymers (e.g., nucleic acids) may both be used. Further, the variety of types and contents of antigens capable of being entrapped may increase. An unlimited modification of lipid compositions also enables features of a particle surface to be changed. Consequently, the application of surface coating and imaging for multifunctional particles is possible.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure. cm What is claimed is:

1. A particle comprising a polymer and a drug that are combined in a liposome formed of a lipid.

2. The particle of claim 1, wherein the polymer is a water-soluble polymer or a lipid-soluble polymer.

3. The particle of claim 2, wherein a concentration of the lipid is in a range of 1 to 10 mM when the polymer is the water-soluble polymer.

4. The particle of claim 2, wherein a concentration of the lipid is in a range of 3 to 5 M when the polymer is the lipid-soluble polymer.

5. The particle of claim 2, wherein the water-soluble polymer is one or more selected from the group consisting of polydeoxyribonucleic acids, agaroses, alginates, carrageenans, hyaluronic acids, dextrans, chitosans, and cyclodextrins.

6. The particle of claim 2, wherein the lipid-soluble polymer is one or more selected from the group consisting of polylactides, polyglycolides, poly-gamma-glutamic acid (BLS-PGA), polycaprolactones, polyethylene glycol, poly(hydroxy butyrate), poly(ε-caprolactone), poly(β-malic acid), poly(lactic acid-co-glycolic acid) and mixtures thereof.

7. The particle of claim 6, wherein the lipid-soluble polymer is one or more selected from the group consisting of polylactides, polyglycolides and mixtures thereof.

8. The particle of claim 7, wherein a mole ratio of a polylactide and a polyglycolide is 25˜75:75˜25 in the mixture.

9. The particle of claim 1, wherein the lipid is one or more selected from the group consisting of 1,2-dioleoyl-sn-glycero-3-phosphocholine (DOPC), 1,2-dioleoyl-sn-glycero-3-phospho-(1′-rac-glycerol) sodium salt (DOPG), 1,2-dioleoyl-sn-glycero-3-phosphoethanolamin-N-[4-(p-maleimidophenyl)butyramide] (MPB-PE), 1,2-dihexadecanoyl-sn-glycero-3-phosphoethanolamine, triethylammonium salt (Texas Red DHPE), cholesterol, lecithin, and mixtures thereof.

10. The particle of claim 1, wherein the drug is ovalbumin or CpG oligodeoxynucleotide.

11. The particle of claim 1, wherein a diameter of the particle is in a range of 200 to 1,500 nm.

12. A method of producing the particle of claim 3, comprising:
a) mixing water-soluble polymers and lipids;
b) preparing an emulsion by stirring the mixed solution or treating the mixed solution with ultrasonic waves; and
c) removing an organic solvent positioned in an upper layer of the emulsion by centrifugation of the emulsion.

13. The method of claim 12, wherein the water-soluble polymer is one or more selected from the group consisting of polydeoxyribonucleic acids, agaroses, alginates, carrageenans, hyaluronic acids, dextrans, chitosans, and cyclodextrins.

14. The method of claim 12, wherein a drug is further mixed in step a).

15. The method of claim 14, wherein the drug is ovalbumin or CpG oligodeoxynucleotide.

16. The method of claim 12, wherein the lipid is one or more selected from the group consisting of 1,2-dioleoyl-sn-glycero-3-phosphocholine (DOPC), 1,2-dioleoyl-sn-glycero-3-phospho-(1′-rac-glycerol) sodium salt (DOPG), 1,2-dioleoyl-sn-glycero-3-phosphoethanolamin-N-[4-(p-maleimidophenyl)butyramide] (MPB-PE), 1,2-dihexadecanoyl-sn-glycero-3-phosphoethanolamine, triethylammonium salt (Texas Red DHPE), cholesterol, lecithin, and mixtures thereof.

17. A method of producing the particle of claim 4, comprising:
a) mixing lipid-soluble polymers and lipids;
b) preparing a single emulsion by treating the mixed solution with ultrasonic waves;
c) preparing a multiple emulsion by adding an aqueous solution including a drug to the single emulsion and treating the single emulsion with ultrasonic waves;
d) removing an organic solvent by stirring the multiple emulsion; and
e) centrifuging the emulsion from which the organic solvent is removed in step d).

18. The method of claim 17, wherein the lipid-soluble polymer is one or more selected from the group consisting of polylactides, polyglycolides, poly-gamma-glutamic acid (BLS-PGA), polycaprolactones, polyethylene glycol, poly(hydroxy butyrate), poly(ε-caprolactone), poly(β-malic acid), poly(lactic acid-co-glycolic acid) and mixtures thereof.

19. The method of claim 18, wherein the lipid-soluble polymer is one or more selected from the group consisting of polylactides, polyglycolides and mixtures thereof.

20. The method of claim 19, wherein a mole ratio of a polylactide and a polyglycolide is 25˜75:75˜25 in the mixture.

21. The method of claim 17, wherein the lipid is one or more selected from the group consisting of 1,2-dioleoyl-sn-glycero-3-phosphocholine (DOPC), 1,2-dioleoyl-sn-glycero-3-phospho-(1′-rac-glycerol) sodium salt (DOPG), 1,2-dioleoyl-sn-glycero-3-phosphoethanolamin-N-[4-(p-maleimidophenyl)butyramide] (MPB-PE), 1,2-dihexadecanoyl-sn-glycero-3-phosphoethanolamine, triethylammonium salt (Texas Red DHPE), cholesterol, lecithin, and mixtures thereof.

22. The method of claim 17, wherein the drug is ovalbumin or CpG oligodeoxynucleotide.
